(19) United States
(12) Patent Application Publication
(10) Pub. No.: US 2006/0014702 A1
(43) Pub. Date: Jan. 19, 2006

(54) Antimicrobial agent

(76) Inventors: Laurence Mark Von Itzstein, Queensland (AU); Christopher Bonner Davis, Queensland (AU); Robin Joy Thomson, Queensland (AU)

Correspondence Address: MOORE & VAN ALLEN PLLC, P.O. BOX 13706, Research Triangle Park, NC, 27709, US

(21) Appl. No.: 10/514,784

(22) PCT Filed: May 22, 2003
(86) PCT No.: PCT/AU03/00620

§ 371 Date: May 25, 2005

(30) Foreign Application Priority Data
May 22, 2002 (AU) PS 2475

(51) Int. Cl. A61K 31/70; C07H 15/00

(52) U.S. Cl. 514/24; 536/4.1

(57) ABSTRACT

The present invention relates to novel thioglycosides of D-galactofuranose that have an antimicrobial action, methods for their synthesis, pharmaceutical compositions containing them and methods for the treatment of patients suffering microbial infection.

### TECHNICAL FIELD

The present invention relates to novel thioglycosides of D-galactofuranose that have an antimicrobial action, methods for their synthesis, pharmaceutical compositions containing them and methods for the treatment of patients suffering microbial infection.

### BACKGROUND ART

Many bacterial diseases once thought to be on the decline are beginning to re-emerge and annually devastate populations in many countries. This problem is amplified by the emergence of many new drug resistant strains of the microorganisms that cause these diseases. The present inventors interest in glycofuranose chemistry (Owen & von Itzstein, 2000) has led to the discovery of a new class of antimicrobial agents described below. Although significant chemistry and biology has been published (see for example: Marino and Lima et al., 2002; Marino et al., 1998; Marino and Marino et al., 2002; Chiocconi et al., 2000; Miletti et al., 1999; Pathak et al., 2002; Pathak et al., 2001; Zhang and Liu, 2001; Brimacombe et al., 1971; Brimacombe et al., 1968; Lemieux and Stick, 1975; Nam Shin and Perlin, 1979; Sznaidman et al., 1986; Cicero et al., 1990; Ernst et al., 2000) in the area of galactofuranose chemistry and biology none to date provides compounds that have significant antimicrobial activity.

### DISCLOSURE OF THE INVENTION

The present invention is concerned generally with novel thioglycosides of D-galactofuranose that have antimicrobial action.

In a first aspect of the present invention there is provided an anti-microbial compound of general formula (I): 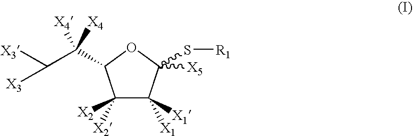
wherein R₁ is selected from the group consisting of optionally substituted alkyl and optionally substituted alkenyl, each of which may be interrupted by one or more heteroatoms or functional groups selected from the group consisting of O, S, —N═, NR₆ and —(Y)ₘC=(Z)(T)ₙ- and contains at least four carbon atoms, and optionally substituted aralkyl which may be interrupted within the alkyl moiety by one or more heteroatoms or functional groups selected from the group consisting of O, S, —N═, NR₆ and —(Y)ₘC=(Z)(T)ₙ-;
X₁ is selected from the group consisting of OR₂, SR₂, NR₂R′₂, halogen, —(Y)ₘC=(Z)(T)ₙR₂, —N(C=(Z)(T)ₙR₂)₂, N₃, CN, OCN, SCN, OSO₃R₂, OSO₂R₂, OPO₃R₂R′₂, OPO₂R₂R′₂, S(O)R₂, S(O)₂R₂, S(O)₂OR₂, PO₃R₂R′₂, NNR₂R′₂, SNR₂R′₂, NHSR₂, SSR₂ and R₂, or is an oxo group, ═S, ═NOR₂ or ═CHR₂ and X₁′ is absent;
X₂ is selected from the group consisting of OR₃, SR₃, NR₃R′₃, halogen, —(Y)ₘC=(Z)(T)ₙR₃, —N(C=(Z)(T)ₙR₃)₂, N₃, CN, OCN, SCN, OSO₃R₃, OSO₂R₃, OPO₃R₃R′₃, OPO₂R₃R′₃, S(O)R₃, S(O)₂R₃, S(O)₂OR₃, PO₃R₃R′₃, NNR₃R′₃, SNR₃R′₃, NHSR₃, SSR₃ and R₃, or is an oxo group, ═S, ═NOR₃ or ═CHR₃ and X₂′ is absent;
X₃ is selected from the group consisting of OR₄, SR₄, NR₄R′₄, halogen, —(Y)ₙC=(Z)(T)ₙR₄, —N(C=(Z)(T)ₙR₄)₂, N₃, CN, OCN, SCN, OSO₃R₄, OSO₂R₄, OPO₃R₄R′₄, OPO₂R₄R′₄, S(O)R₄, S(O)₂R₄, S(O)₂OR₄, PO₃R₄R′₄, NNR₄R′₄, SNR₄R₁₄, NHSR₄, SSR₄ and R₄, or is an oxo group, ═S, ═NOR₄ or ═CHR₄ and X₃′ is absent;
X₄ is selected from the group consisting of OR₅, SR₅, NR₅R′₅, halogen, —(Y)ₘC=(Z)(T)ₙR₅, —N(C=(Z)(T)ₙR₅)₂, N₃, CN, OCN, SCN, OSO₃R₅, OSO₂R₅, OPO₃R₅R′₅, OPO₂R₅R′₅, S(O)R₅, S(O)₂R₅, S(O)₂OR₅, PO₃R₅R′₅, NNR₅R′₅, SNR₅R′₅, NHSR₅, SSR₅ and R₅, or is an oxo group, ═S, ═NOR₅ or ═CHR₅ and X₄′ is absent;
or X₁ and X₂ together constitute a double bond, or X₁ and X₂, X₂ and X₃, X₂ and X₄, X₃ and X₄, X₁, and X₁′, X₂ and X₂′, X₃ and X₃′ or X₄ and X₄′ together form a ring;
m and n are independently zero or one and Y, Z and T are independently selected from the group consisting of O, S, and NR₆ where R₆ is hydrogen or alkyl;
X₅, X₁′, X₂′, X₃′ and X₄′ are the same or different and are selected from the group consisting of hydrogen, CN, optionally substituted alkyl, optionally substituted alkaryl, optionally substituted aryl, and optionally substituted aralkyl;
R₂, R′₂, R₃, R′₃, R₄, R′₄, R₅ and R′₅ are the same or different and are selected from the group consisting of hydrogen, optionally substituted alkyl, optionally substituted alkaryl, optionally substituted aryl, optionally substituted aralkyl, optionally substituted acyl and a carbohydrate moiety;
with the proviso that R₁ is other than benzyl and at least two of X₁, X₂, X₃ and X₄ are other than hydrogen or a group linked to the ring through a carbon-carbon bond;
or a pharmaceutically acceptable salt thereof.

The term “alkyl” used either alone or in a compound word such as “optionally substituted alkyl” or “optionally substituted cycloalkyl” denotes straight chain, branched or mono- or poly-cyclic alkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimetylpentyl, 1,2-dimethylpentyl, 1,3-dimethylpentyl, 1,4-dimethylpentyl, 1,2,3-trimethylbutyl, 1,1,2-trimethylbutyl, nonyl, 1-, 2-, 3-, 4-, 5-, 6- or 7-methyloctyl, 1-, 2-, 3-, 4- or 5-ethylheptyl, 1-2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Examples of cycloalkyl include cyclopropyl, cyclobutyl, cyclopentyl, cyclohexyl, cycloheptyl, cyclooctyl, cyclononyl and cyclodecyl and the like.

The term “alkenyl” used either alone or in compound words such as “alkenyloxy” denotes groups formed from straight chain, branched or cyclic alkenes including ethylenically mono-, di- or poly-unsaturated alkyl or cycloalkyl groups as defined above. Examples of alkenyl with at least 4 carbon atoms include butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1-4, pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl.

The term “acyl” used either alone or in compound words such as “optionally substituted acyl” or “optionally substituted acyloxy” denotes an aliphatic acyl group or an acyl group containing an aromatic ring, which is referred to as aromatic acyl, or a heterocyclic ring, which is referred to as heterocyclic acyl, preferably C₁₋₃₀ acyl. Examples of acyl include straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutyl, phenylpentanoyl and phenylhexanoyl) and naphthylalkanoyl (e.g. naphthylacetyl, naphthylpropanoyl and naphthylbutanoyl); aralkenoyl such as phenylalkenoyl (e.g. phenylpropenoyl, phenylbutenoyl, phenylmethacrylyl, phenylpentenoyl and phenylhexenoyl and naphthylalkenoyl (e.g. naphthylpropenoyl, naphthylbutenoyl and naphthylpentenoyl); heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; and heterocyclicalkenoyl such as heterocyclicpropenoyl, heterocyclicbutenoyl, heterocyclicpentenoyl and heterocyclichexenoyl.

The term “aryl” used either alone or in compound words such as “optionally substituted aryl”, “optionally substituted aryloxy” or “optionally substituted heteroaryl” denotes single, polynuclear, conjugated and fused residues of aromatic hydrocarbons or aromatic heterocyclic ring systems. Examples of aryl include phenyl, biphenyl, terphenyl, quaterphenyl, phenoxyphenyl, naphtyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl, pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Preferably, the aromatic heterocyclic ring system contains 1 to 4 heteroatoms independently selected from N, O and S and containing up to 9 carbon atoms in the ring.

The term “heterocyclyl” used either alone or in compound words such as “optionally substituted saturated or unsaturated heterocyclyl” denotes monocyclic or polycyclic heterocyclyl groups containing at least one heteroatom atom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl; saturated 3 to 6-membered heteromonocyclic groups containing 1 to 4 nitrogen atoms, such as, pyrrolidinyl, imidazolidinyl, piperidino or piperazinyl; unsaturated condensed heterocyclic groups containing 1 to 5 nitrogen atoms, such as indolyl, isoindolyl, indolizinyl, benzimidazolyl, quinolyl, isoquinolyl, indazolyl, benzotriazolyl or tetrazolopyridazinyl; unsaturated 3 to 6-membered heteromonocyclic group containing an oxygen atom, such as, pyranyl or furyl; unsaturated 3 to 6-membered heteromonocyclic group containing 1 to 2 sulfur atoms, such as, thienyl; unsaturated 3 to 6-membered heteromonocyclic group containing 1 to 2 oxygen atoms and 1 to 3 nitrogen atoms, such as, oxazolyl, isoxazolyl or oxadiazolyl; saturated 3 to 6-membered heteromonocyclic group containing 1 to 2 oxygen atoms and 1 to 3 nitrogen atoms, such as, morpholinyl; unsaturated condensed heterocyclic group containing 1 to 2 oxygen atoms and 1 to 3 nitrogen atoms, such as, benzoxazolyl or benzoxadiazolyl; unsaturated 3 to 6-membered heteromonocyclic group containing 1 to 2 sulphur atoms and 1 to 3 nitrogen atoms, such as, thiazolyl or thiadiazolyl; saturated 3 to 6-membered heteromonocyclic group containing 1 to 2 sulphur atoms and 1 to 3 nitrogen atoms, such as, thiazolidinyl; and unsaturated condensed heterocyclic group containing 1 to 2 sulphur atoms and 1 to 3 nitrogen atoms, such as, benzothiazolyl or benzothiadiazolyl.

The term “carbohydrate” used either alone or in compound words such as “optionally substituted carbohydrate” denotes a carbohydrate residue or a further functionalised carbohydrate residue containing a 5- or 6-membered ring that may be substituted, for example, by alkyl or acyl groups, and includes monosaccharides and oligosaccharides. Examples of carbohydrates include but are not limited to D-galactofuranose, N-acetyl-D-galactofuranose, D-glucofuranose, N-acetyl-D-glucofuranose, D-galactopyranose N-acetyl-D-galactopyranose, D-glucopyranose and N-acetyl-D-glucopyranose as well as oligosaccharides containing these moieties.

In this specification “optionally substituted” means that a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, benzylthio, acylthio, phosphorus-containing groups and the like, provided that none of the substituents outlined above interferes with the formation of the subject compound or renders it biologically inactive.

Any of the moieties whose length is defined in terms of the number of carbon atoms present may possess any number of carbon atoms within the specified range. Nevertheless, within this range certain species will be preferred due to factors such as availability and cost of precursors and ease of synthesis, as well as efficacy. In particular, such moieties containing 4 to 30 carbon atoms, preferably 6 to 16 carbon atoms, more preferably 6, 8, 10 or 16 carbon atoms and most preferably 10 carbon atoms are preferred for reasons of cost and availability of precursors, ease of synthesis and efficacy.

In one form of the invention, R₁ is n-alkyl which is optionally substituted and may include a heteroatom or a functional group, for example an acyl linkage, in the alkyl chain. Preferably, R₁ is C₄₋₃₀ alkyl, more preferably C₆₋₁₆ alkyl, most preferably C₁₀ alkyl.

Alternatively, R₁ is a branched alkyl moiety which is optionally substituted and may include a heteroatom or a functional group, for example an acyl linkage, in the alkyl chain. In a preferred embodiment of the invention R₁ takes general formula (II): 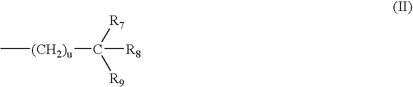
wherein u is 0 to 22, preferably 0 to 10, more preferably 0 to 4, more preferably still 0 to 2, and most preferably 0 and wherein a heteroatom or acyl linkage may separate adjacent CH₂ groups within the moiety; and
at least two of R₇, R₈ and R₉ are alkyl, typically n-alkyl, and the other is hydrogen or alkyl, typically hydrogen. Preferably, each alkyl moiety is C₂₋₃₀ alkyl, more preferably C₄₋₂₄ alkyl, still more preferably C₆₋₁₆ alkyl, most preferably C₁₀ alkyl, and may be the same or different.

In a particularly preferred embodiment of the invention, R₁ takes the general formula (III): 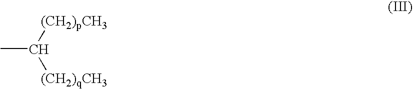
wherein p and q are the same or different and each is ≧1, preferably 1-29, more preferably 3-23, still more preferably 5-15, and most preferably 9.

As an example of a heteroalkyl chain, R₁ takes the general (Iv): 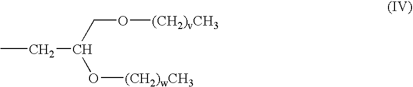
wherein v and w are the same or different and each is ≧1, preferably 1-29, more preferably 3-23, still more preferably 5-15, and most preferably 9.

X₁, X₂, X₃ and X₄ may be any combination of substituents, but at least two of these moieties should be other than hydrogen or a group linked to the ring through a carbon-carbon bond. Preferably, at least two of X₁, X₂, X₃ and X₄ are moieties linked to the ring through a carbon-oxygen bond, for example, in the case of X₁, OR₂, —(Y)ₘC=(Z)(T)ₙR₂ when Y is O, OSO₃R₂ and OPO₃R₂R′₂. The group —(Y)ₘC=(Z)(T)ₙR₂ is preferably —OC(O)R₂.

Preferably X₁ is OR₂ or OC(O)R₂. Advantageously X₁ is hydroxyl or acyloxy, preferably C₁₋₃₀ acyloxy, more preferably hydroxyl, acetyloxy or benzoyloxy.

Preferably X₂ is OR₃ or OC(O)R₃. Advantageously X₂ is hydroxyl or acyloxy, preferably C₁₋₃₀ acyloxy, more preferably hydroxyl, acetyloxy or benzoyloxy.

Preferably X₃ is OR₄ or OC(O)R₄. Advantageously X₃ is hydroxyl or acyloxy, preferably C₁₋₃₀ acyloxy, more preferably hydroxyl, acetyloxy or benzoyloxy

Preferably X₄ is OR₅ or OC(O)R₅. Advantageously X₄ is hydroxyl or acyloxy, preferably C₁₋₃₀ acyloxy, more preferably hydroxyl, acetyloxy or benzoyloxy.

Advantageously the compound of general formula (I) is selected from the group consisting of hexadecyl 2,3,5,6-tetra-O-benzoyl-1-thio-β-D-galactofuranoside, decyl 2,3,5,6-tetra-O-benzoyl-1-thio-β-D-galactofuranoside, octyl 2,3,5,6-tetra-O-benzoyl-1-thio-β-D-galactofuranoside, hexyl 2,3,5,6-tetra-O-benzoyl-1-thio-β-D-galactofuranoside, 11-heneicosanyl 2,3,5,6-tetra-O-benzoyl-1-thio-β-D-galactofuranoside, hexadecyl 1-thio-β-D-galactofuranoside, decyl 1-thio-β-D-galactofuranoside, octyl 1-thio-β-D-galactofuranoside, hexyl 1-thio-β-D-galactofuranoside, and 11-heneicosanyl 1-thio-β-D-galactofuranoside.

In a particularly preferred embodiment of the invention the compound of general formula (I) is hexadecyl 1-thio-β-D-galactofuranoside, decyl 1-thio-β-D-galactofuranoside, octyl 1-thio-β-D-galactofuranoside, hexyl 1-thio-β-D-galactofuranoside, or 11-heneicosanyl 1-thio-β-D-galactofuranoside, most particularly decyl 1-thio-β-D-galactofuranoside.

According to a second aspect of the present invention there is provided a method of preparation of a compound of general formula (I): 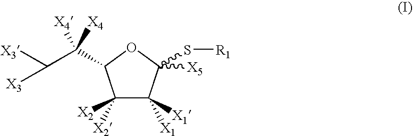
comprising reacting a compound of general formula (V): 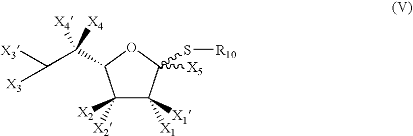
wherein R₁₀ is an acyl group, preferably acetyl and X₁, X₂, X₃ and X₄ are as defined above, but with any free hydroxyl, thiol, or amine groups protected by a protecting group;
with a compound of general formula (VI):
   Br—R₁ (VI)
wherein R₁ is as defined above;
in the presence of a base;
   and, optionally
removing the protecting groups.

Typically the base is diethylamine (Bennet et al., 1994) or hydrazinium acetate/triethylamine (Park et al., 1995). In general terms the reaction is performed in the presence of an excess of the base in an inert solvent such as DMF or THF, or mixtures of such solvents, at a temperature from 20° C. to 60° C., preferably 25-40° C., under an atmosphere of nitrogen or argon. The reaction mixture may be left to stir typically for 1 to 24 hours, preferably 2 to 6 hours, most preferably 4 hours, prior to isolation and purification, or deprotection. Suitable protecting groups are well known to a person skilled in the art and in this case the benzoyl group is preferred. Benzoyl protecting groups are typically removed through hydrolysis with sodium methoxide in methanol. The compounds of the present invention may also be synthesised through reaction of a compound of general formula (V) in the presence of base, with a sulfonate ester of the alcohol corresponding to bromide (VI), or via the relevant carbohydrate C-1 thiols [compound of general formula (V) where R₁₀ is H] in the presence of base with a compound of general formula (VI) or a sulfonate ester of the corresponding alcohol. The compounds of the present invention may also be synthesised through reaction of a compound of general formula (VII): 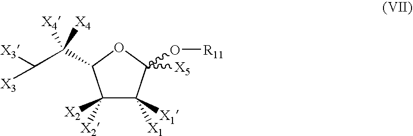
wherein R₁₁ is an acyl group, preferably acetyl or benzoyl, and X₁, X₂, X₃ and X₄ are as defined above but with any free hydroxyl, thiol, or amine groups protected by a protecting group;
with a compound of general formula (VIII):
   HS—R₁ (VIII)
wherein R₁ is as defined above;
in the presence of a catalyst, typically a Lewis acid;
   and, optionally
removing the protecting groups.

Typically the Lewis acid is tin tetrachloride (Marino et al., 1998). In general terms the reaction is performed in the presence of a slight excess of the Lewis acid in an inert solvent such as dichloromethane, at a temperature of 0° C., under an atmosphere of nitrogen or argon. The reaction mixture is left to stir typically for 2 hours, prior to isolation and purification, or deprotection. Methods for the preparation of compounds of general formulae (VI) and (VIII) are well known to a person skilled in the art. An extensive array of methodologies has been developed to manipulate each position of the galactofuranose template as disclosed, for example, in Marino et al., 1998; Marino and Marino et al., 2002; Chiocconi et al., 2000; Miletti et al., 1999; Zhang and Liu, 2001; Brimacombe et al., 1971; Brimacombe et al., 1968; Lemieux and Stick, 1975; Nam Shin and Perlin, 1979; Sznaidman et al., 1986; Cicero et al., 1990; Ernst et al., 2000; the contents of which are incorporated herein by reference.

According to a third aspect of the present invention there is provided a method for the treatment of a patient with a microbial infection, comprising administering to said patient a therapeutically effective amount of a compound of general formula (I).

According to a fourth aspect of the present invention there is provided the use of a compound of general formula (I) in the manufacture of a medicament for use in the treatment of a microbial infection.

As used herein, the term “therapeutically effective amount” means an amount of a compound of the present invention effective to yield a desired therapeutic response, for example to prevent or treat a disease which by administration of a pharmaceutically-active agent.

The specific “therapeutically effective amount” will, obviously, vary with such factors as the particular condition being treated, the physical condition and clinical history of the subject, the type of animal being treated, the duration of the treatment, the nature of concurrent therapy (if any), and the specific formulations employed and the structure of the compound or its derivatives.

As used herein, a “pharmaceutical carrier” is a pharmaceutically acceptable solvent, suspending agent, excipient or vehicle for delivering the compound of general formula (I) to the subject. The carrier may be liquid or solid, and is selected with the planned manner of administration in mind.

The compound of general formula (I) may be administered orally, topically, or parenterally in dosage unit formulations containing conventional non-toxic pharmaceutically acceptable carriers, adjuvants, and vehicles. The term parenteral as used herein includes subcutaneous, intravenous, intramuscular, intrathecal, intracranial, injection or infusion techniques.

The invention also provides suitable topical, oral, aerosol, and parenteral pharmaceutical formulations for use in the novel methods of treatment of the present invention. The compounds of the invention may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. The tablets contain the active ingredient in admixture with non-toxic pharmaceutically acceptable excipients which are suitable for the manufacture of tablets.

These excipients may be, for example, inert diluents, such as calcium carbonate, lactose, calcium phosphate or sodium phosphate; granulating and disintegrating agents, such as corn starch or alginic acid; binding agents, such as starch, gelatin or acacia; or lubricating agents, such as magnesium stearate, stearic acid or talc. The tablets may be uncoated, or may be coated by known techniques to delay disintegration and absorption in the gastrointestinal tract and thereby provide a sustained action over a longer period. For example, a time-delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The compound of general formula (I) of the invention can be administered, for in vivo application, parenterally by injection or by gradual perfusion over time independently or together. Administration may be intravenously, intra-arterial, intraperitoneally, intramuscularly, subcutaneously, intracavity, or transdermally. For in vitro studies the agents may be added or dissolved in an appropriate biologically acceptable buffer and added to a cell or tissue.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's intravenous vehicles include fluid and nutrient replenishers, electrolyte replenishers such as those based on Ringer's dextrose, and the like. Preservatives and other additives may also be present such as, for example, anti-microbials, anti-oxidants, chelating agents, growth factors and inert gases and the like.

The compounds of general formula (I) are antimicrobial agents which are active, in particular but not limited to, against Mycobacterium including Mycobacterium smegmatis, Staphylococcus including Staphylococcus aureus, and Enterococci species. The compounds of general formula (I) are particularly useful in treating infections involving these organisms.

Generally, the terms “treating”, “treatment” and the like are used herein to mean affecting a subject, tissue or cell to obtain a desired pharmacological and/or physiological effect. The effect may be prophylactic in terms of completely or partially preventing infection, and/or may be therapeutic in terms of a partial or complete cure of an infection. “Treating” as used herein covers any treatment of, or prevention of infection in a vertebrate, a mammal, particularly a human, and includes: preventing the infection from occurring in a subject that may have been exposed to the infectious agent, but has not yet been diagnosed as affected; inhibiting the infection, ie. arresting its development; or relieving or ameliorating the effects of the infection, ie., cause regression of the effects of the infection.

According to a fifth aspect of the present invention there is provided a pharmaceutical composition comprising a compound of general formula (I) and a pharmaceutically acceptable carrier.

The pharmaceutical compositions according to one embodiment of the invention are prepared by bringing a compound of general formula (I) into a form suitable for administration to a subject using carriers, excipients and additives or auxiliaries.

Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

The pharmaceutical compositions are preferably prepared and administered in dosage units. Solid dosage units include tablets, capsules and suppositories. For treatment of a subject, depending on activity of the compound, manner of administration, nature and severity of the disorder, age and body weight of the subject, different daily doses can be used. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The pharmaceutical compositions according to the invention may be administered locally or systemically in a therapeutically effective dose. Amounts effective for this use will, of course, depend on the severity of the microbial infection and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, eg., in Langer, Science, 249: 1527, (1990). Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be suspending agents such as sodium carboxymethyl cellulose, methyl cellulose, hydroxypropylmethylcellulose, sodium alginate, polyvinylpyrrolidone, gum tragacanth and gum acacia; dispersing or wetting agents, which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

The pharmaceutical compositions may be in the form of a sterile injectable aqueous or oleaginous suspension. This suspension may be formulated according to known methods using suitable dispersing or wetting agents and suspending agents such as those mentioned above. The sterile injectable preparation may also a sterile injectable solution or suspension in a non-toxic parenterally-acceptable diluent or solvent, for example, as a solution in 1,3-butanediol. Among the acceptable vehicles and solvents which may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

Compounds of general formula (I) may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Dosage levels of the compound of general formula (I) of the present invention will usually be of the order of about 0.05 mg to about 20 mg per kilogram body weight, with a preferred dosage range between about 0.05 mg to about 10 mg per kilogram body weight per day (from about 0.1 g to about 3 g per patient per day). The amount of active ingredient which may be combined with the carrier materials to produce a single dosage will vary, depending upon the host to be treated and the particular mode of administration. For example, a formulation intended for oral administration to humans may contain about 1 mg to 1 g of an active compound with an appropriate and convenient amount of carrier material, which may vary from about 5 to 95 percent of the total composition. Dosage unit forms will generally contain between from about 5 mg to 500 mg of active ingredient.

It will be understood, however, that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

In addition, some of the compounds of the invention may form solvates with water or common organic solvents. Such solvates are encompassed within the scope of the invention.

The compounds of the invention may additionally be combined with other compounds to provide an operative combination. It is intended to include any chemically compatible combination of pharmaceutically-active agents, as long as the combination does not eliminate the activity of the compound of general formula (I) of this invention.

According to a sixth aspect of the present invention there is provided a method of killing a microorganism, comprising exposing said microorganism to a compound of general formula (I) as defined above.

Advantageously, although not limited to, the microorganism is selected from the group consisting of Mycobacterium including Mycobacterium smegmatis, Staphylococcus including Staphylococcus aureus, and Enterococci species.

Throughout this specification and the claims, the words “comprise”, “comprises” and “comprising” are used in a non-exclusive sense, except where the context requires otherwise.

It will be clearly understood that, although a number of prior art publications are referred to herein, this reference does not constitute an admission that any of these documents forms part of the common general knowledge in the art, in Australia or in any other country.

### MODES FOR PERFORMING THE INVENTION

The synthetic scheme employed to prepare compounds in accordance with preferred embodiments of the invention is now described in more detail. 1-S-Acetyl-2,3,5,6-tetra-O-benzoyl-1-thio-β-D-galactofuranose (compound 2) was prepared according to known literature methods (Owen and von Itzstein, 2000) without modification and is shown in Scheme 1. All new compounds gave the expected spectroscopic data. The synthesis of protected 3 (Examples 1, 2, 3, and 4) and deprotected 4 (Examples 5, 6 and 7) alkyl 1-thio-β-D-galactofuranosides is described in Scheme 1. 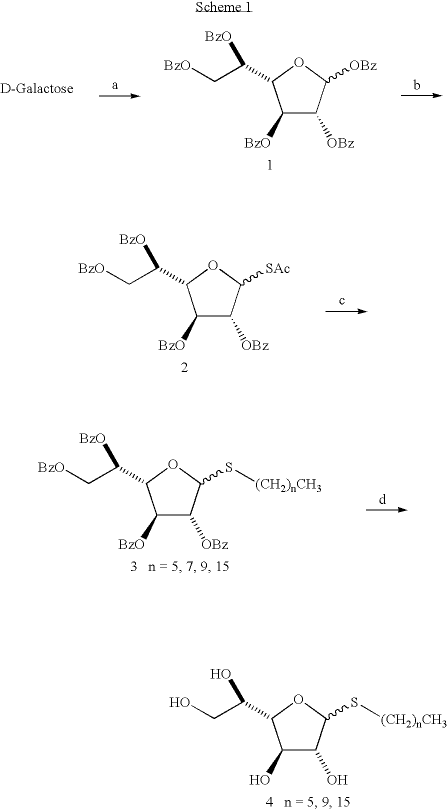
Reagents and Conditions: a) i) pyr, 100° C., 1 h, ii) BzCl, 60° C., 2 h, iii) rt, 24 h; b) SnCl₄, CH₂Cl₂, HSAc, rt, 1 h, N₂; c) Br(CH₂)ₙCH₃, DMF, HN(CH₂CH₃)₂, rt, 4 h, N₂; d) NaOMe, MeOH, rt, 2 h, N₂.

The synthesis of protected 5 (Example 8) and deprotected 6 (Example 9) branched alkyl 1-thio-β-D-galactofuranosides is described in Scheme 2. 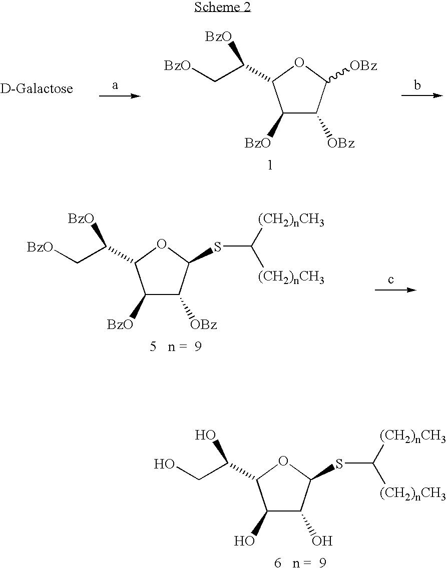
Reagents and Conditions: a) i) pyr, 100° C., 1 h, ii) BzCl, 60° C., 2 h, iii) rt, 24 h; b) SnCl₄, CH₂Cl₂, HSC[(CH₂)ₙCH₃]₂, 0° C., 2 h, N₂; c) NaOMe, MeOH, rt, 2 h, N₂.

### EXAMPLE 1

### Hexadecyl 2,3,5,6-tetra-O-benzoyl-1-thio-β-D-galactofuranoside (3, n=15)

To a solution of 1-S-acetyl-2,3,5,6-tetra-O-benzoyl-1-thio-β-D-galactofuranose 2 (440 mg, 0.67 mmol) and 1-bromohexadecane (205 μl, 0.67 mmol) in dry DMF (5 ml) under N₂ was added diethylamine (1.5 ml, excess). The reaction was left to stir at room temperature for 4 h. After this time the diethylamine and DMF were removed under reduced pressure. The residue was then diluted in EtOAc (50 ml), washed once with 0.5 M HCl (50 ml) and twice with water (50 ml) and dried over Na₂SO₄. The solvent was removed under reduced pressure and the residue chromatographed to give 3 (n=15) as a pale brown syrup (281 mg, 50%). R_{f} 0.51 (hexane-EtOAc 6:1); ¹H NMR (300 MHz, CDCl₃): δ 7.26-8.10 (m, 20H, 4×OCOPh), 6.08 (m, 1H, H-5), 5.66 (apparent d, 1H, J_{3,4} 5.1 Hz, H-3), 5.63 (broad s, 1H, H-1), 5.50 (apparent t, 1H, J_{2,3}=J_{2,1} 1.4 Hz, H-2), 4.83 (apparent triplet, 1H, J 4.1, 4.7 Hz, H-4), 4.75 (m, 2H, H-6a and H-6b), 2.69 (m, 2H, SCHa and SCHb), 1.65 (apparent quintet, 2H, J 7.5 Hz, S—CH₂—CHa and S—CH₂—CHb), 1.15-1.40 (m, 26H, 13×CH₂), 0.88 (t, 3H, J 6.4 Hz, CH₃); ¹³C NMR (75.5 MHz, CDCl₃): δ 166.0, 165.7, 165.5, 165.4 (4×OCOPh), 133.5, 133.4, 133.2, 133.0, 130.0, 130.0, 130.0, 129.8, 129.7, 129.5, 129.5, 129.0, 128.9, 128.4, 128.4, (4×OCOPh), 88.3 (C1), 82.9 (C2), 81.0 (C4), 77.9 (C3), 70.3 (C5), 63.4 (C6), 31.9 (S—CH₂), 31.2 (S—CH₂—CH₂), 29.7, 29.6, 29.6, 29.3, 29.2, 28.9, 22.7, (13×CH₂), 14.1 (CH₃); LRMS (ESI): m/z 859 [(M+Na)⁺, (100%)], 875 (13), 854 (11).

### EXAMPLE 2

### Decyl 2,3,5,6-tetra-O-benzoyl-1-thio-β-D-galactofuranoside (3, n=9)

To a soln. of 1-S-acetyl-2,3,5,6-tetra-O-benzoyl-1-thio-β-D-galactofuranose 2 (820 mg, 1.25 mmol) and 1-bromodecane (260 μl, 1.25 mmol) in dry DMF (8 ml) under N₂ was added diethylamine (3 ml, excess). The reaction was left to stir at room temperature for 4 h. After this time the diethylamine and DMF were removed under reduced pressure. The residue was then diluted in EtOAC (50 ml), washed once with 0.5 M HCl (50 ml) and twice with water (50 ml) and dried over Na₂SO₄. The solvent was removed under reduced pressure and the residue chromatographed to give 3 (n=9) as a pale brown syrup (409 mg, 43%). R_{f} 0.45 (hexane-EtOAc 6:1); ¹H NM (300 MHz, CDCl₃): δ 7.26-8.12 (m, 20H, 4×OCOPh), 6.11 (m, 1H, H-5), 5.69 (apparent d, 1H, J_{3,4} 5.2 Hz, H-3), 5.63 (broad s, 1H, H-1), 5.53 (apparent t, 1H, J_{2,3}=J_{2,1} 1.4 Hz, H-2), 4.85 (apparent t, 1H, J 4.2, 4.6 Hz, H-4), 4.78 (m, 2H, H-6a and H-6b), 2.70 (m, 2H, SCHa and SCHb), 1.65 (apparent quintet, 2H, J 7.5 Hz, S—CH₂—CHa and S—CH₂—CHb), 1.20-1.42 (m, 14H, 7×CH₂), 0.89 (apparent t, 3H, J 6.7 Hz, CH₃); ¹³C NMR (75.5 MHz, CDCl₃): δ 166.3, 166.0, 165.8, 165.7 (4×OCOPh), 133.8, 133.6, 133.5, 133.3, 130.3, 130.2, 130.1, 130.0, 129.8, 129.7, 129.2, 129.2, 128.7, 128.7, 128.6 (4×OCOPh), 88.8 (C1), 83.1 (C2), 81.3 (C4), 78.2 (C3), 70.5 (C5), 63.7 (C6), 32.2 (S—CH₂), 31.5 (S—CH₂—CH₂), 29.9, 29.8, 29.8, 29.5, 29.4, 29.1, 22.9 (7×CH₂), 14.3 (CH₃).

### EXAMPLE 3

### Octyl 2,3,5,6-tetra-O-benzoyl-1-thio-β-D-galactofuranoside (3, n=7)

To a solution of 1-S-acetyl-2,3,5,6-tetra-O-benzoyl-1-thio-β-D-galactofuranose 2 (464 mg, 0.71 mmol) and 1-bromooctane (115 μl, 0.67 mmol) in dry DMF (5 ml) under N₂ was added diethylamine (2.0 ml, excess). The reaction was left to stir at room temperature for 4 h. After this time the diethylamine and DMF were removed under reduced pressure. The residue was then diluted in EtOAc (50 ml), washed once with 0.5 M HCl (50 ml) and twice with water (50 ml) and dried over Na₂SO₄. The solvent was removed under reduced pressure and the residue chromatographed to give 3 (n=7) as a pale brown syrup (159 mg, 30%). R_{f} 0.35 (hexane-EtOAc 4:1); ¹H NMR (300 MHz, CDCl₃): δ 7.26-8.10 (m, 20H, 4×OCOPh), 6.10 (m, 1 H, H-5), 5.69 (dd, 1H, J_{3,4} 4.1, J_{3,2} 0.9 Hz, H-3), 5.63 (broad s, 1H, H-1), 5.57 (apparent t, 1H, J_{2,3}=J_{2,1} 1.4 Hz, H-2), 4.84 (dd, 1H, J_{4,5} 4.6, J_{4,3} 3.9 Hz, H-4), 4.74 (m, 2H, H-6a and H-6b), 2.68 (m, 2H, SCHa and SCHb), 1.64 (apparent quintet, 2H, J 7.5 Hz, S—CH₂—CHa and S—CH₂—CHb), 1.15-1.45 (m, 10H, 5×CH₂) 0.89 (apparent t, 3H, J 6.9 Hz, CH₃); ¹³C NMR (75.5 MHz, CDCl₃): δ 166.5, 166.1, 166.0, 165.8 (4×OCOPh), 133.9, 133.8, 133.7, 133.5, 130.4, 130.4, 130.3, 130.1, 130.0, 129.9, 129.4, 129.3, 128.9, 128.8, 128.8, 128.7, (4×OCOPh), 88.9 (C1), 83.3 (C2), 81.5 (C4), 78.3 (C3), 70.7 (C5), 63.9 (C6), 32.2 (S—CH₂), 31.6 (S—CH₂—CH₂), 30.1, 29.6, 29.5, 29.3, 23.0 (5×CH₂), 14.5 (CH₃): LRMS (ESI): m/z 747 [(M+Na)⁺, (97%)], 763 (20), 579 (100).

### EXAMPLE 4

### Hexyl 2,3,5,6-tetra-O-benzoyl-1-thio-β-D-galactofuranoside (3, n=5)

To a solution of 1-S-acetyl-2,3,5,6-tetra-O-benzoyl-1-thio-β-D-galactofuranose 2 (807 mg, 1.23 mmol) and 1-bromohexane (173 μl, 1.23 mmol) in dry DMF (5 ml) under N₂ was added diethylamine (3.0 ml, excess). The reaction was left to stir at room temperature for 4 h. After this time the diethylamine and DMF were removed under reduced pressure. The residue was then diluted in EtOAc (50 ml), washed once with 0.5 M HCl (50 ml) and twice with water (50 ml) and dried over Na₂SO₄. The solvent was removed under reduced pressure and the residue chromatographed to give 3 (n=5) as a pale brown syrup (363 mg, 42%). R_{f} 0.31 (hexane-EtOAc 6:1); ¹H NMR (300 MHz, CDCl₃): δ 7.26-8.12 (m, 20H, 4×OCOPh), 6.09 (m, 1H, H-5), 5.67 (apparent d, 1H, J_{3,4} 5.2 Hz, H-3), 5.63 (broad s, 1H, H-1), 5.55 (apparent t, 1H, J_{2,3}=J_{2,1} 1.4 Hz, H-2), 4.83 (apparent t, 1H, J 4.6, 4.2 Hz, H-4), 4.75 (m, 2H, H-6a and H-6b), 2.69 (m, 2H, SCHa and SCHb), 1.65 (apparent quintet, 2H, J 7.5 Hz, S—CH₂—CHa and S—CH₂—CHb), 1.15-1.45 (m, 6H, 3×CH₂), 0.89 (apparent t, 3H, J 6.7 Hz, CH₃). ¹³C NMR (75.5 MHz, CDCl₃); δ 166.3, 166.0, 165.8, 165.7 (4×OCOPh), 133.8, 133.6, 133.5, 133.3, 130.3, 130.3, 130.2, 130.2, 130.1, 130.0, 129.9, 129.8, 129.7, 129.2, 129.2, 128.9, 128.7, 128.7, 128.6, 128.6, 128.5, 128.5 (4×OCOPh), 88.7 (C1), 83.1 (C2), 81.3 (C4), 78.2 (C3), 70.5 (C5), 63.7 (C6), 31.6 (S—CH₂), 31.5 (S—CH₂—CH₂), 29.9, 28.8, 22.8, (3×CH₂), 14.2 (CH₃); LRMS (ESI): m/z 719 [(M+Na)⁺, (60%)], 735 (7), 579 (100).

General Procedure for the Removal of Benzoate Protecting Groups:

To a solution of the protected alkyl 1-thioglycoside (0.45 mmol) in dry and degassed methanol (20 mL) under an atmosphere of N₂ was added one equivalent of sodium methoxide (1M solution in dry and degassed methanol). The reaction was left to stir at room temperature for 2 h. After this time the reaction was neutralised with Amberlite (H⁺) resin. The resin was removed by filtration, the solvent evaporated under reduced pressure and the residue chromatographed to furnish the desired deprotected compound.

### EXAMPLE 5

### Hexadecyl 1-thio-β-D-galactofuranoside (4, n=15)

Yield: 81%. R_{f} 0.50 (EtOAc); ¹H NMR (300 MHz, CD₃OD): δ 4.99 (d, 1H, J_{1,2} 4.5 Hz, H-1), 4.01 (dd, 1H, J_{3,4} 7.4, J_{3,2} 4.8 Hz, H-3), 3.88 (dd, 1H, J_{4,5} 3.2, J_{4,3} 7.4 Hz, H-4), 3.84 (apparent t, 1H, J_{2,3}=J_{2,1} 4.7 Hz, H-2), 3.69 (m, 1H, H-5), 3.57 (d, 2H, J 6.2 Hz, H-6a and H-6b), 2.60 (m, 2H, SCHa and SCHb), 1.60 (apparent quintet, 2H, J 7.4 Hz, SCH₂CHa and SCH₂CHb), 1.18-1.42 (m, 26H, 3×CH₂), 0.86 (t, 3H, J 6.8 Hz, CH₃).

### EXAMPLE 6

### Decyl 1-thio-β-D-galactofuranoside (4, n=9)

Yield: 74%. R_{f} 0.73 (EtOAc-EtOH 7:2) ¹H NMR (300 MHz, CD₃OD): δ 5.02 (d, 1H, J_{1,2} 4.6 Hz, H-1), 4.03 (dd, 1H, J_{3,4} 7.6, J_{3,2} 5.0 Hz, H-3), 3.90 (dd, 1H, J_{4,5} 2.9, J_{4,3} 7.6 Hz, H-4), 3.87 (app t, 1H, J_{2,3}=_{2,1} 4.8 Hz, H-2), 3.72 (m, 1H, H-5), 3.60 (d, 2H, J 6.5 Hz, H-6 and H-6′), 2.63 (m, 2H, SCH₂), 1.62 (m, 2H, J 7.4 Hz, SCH₂CH₂), 1.22-1.46 (m, 14H, 7×CH₂, decyl chain), 0.89 (app t, 3H, J 6.5, 7.0 Hz, CH₃); ¹³C NMR (75.5 MHz, CD₃OD): δ 90.7 (C-1), 83.9 (C-2), 82.8 (C-4), 78.3 (C-3), 72.1 (C-5), 64.6 (C-6), 33.1, 32.0, 31.1, 30.7, 30.5, 30.4, 30.0 (9×CH₂, decyl chain), 14.5 (2×CH₃); LRMS (ESI) m/z 359 [(M+Na)⁺ 33%] 242 (100).

### EXAMPLE 7

### Hexyl 1-thio-β-D-galactofuranoside (4, n=5)

Yield: 97%. R_{f} 0.38 (EtoAc); ¹H NMR (300 MHz, CD₃OD): δ 4.99 (d, 1H, J_{1,2} 4.5 Hz, H-1), 4.01 (dd, 1H, J_{3,4} 7.5, J_{3,2} 4.9 Hz, H-3), 3.88 (dd, 1H, J_{4,5} 4.9, J_{4,3} 7.5 Hz, H-4), 3.85 (apparent t, 1H, J_{2,3}=J_{2,1} 4.7 Hz, H-2), 3.69 (m, 1H, H-5), 3.57 (d, 2H, J 6.2 Hz, H-6a and H-6b), 2.60 (m, 2H, SCHa and SCHb), 1.59 (apparent quintet, 2H, J 7.6 Hz, SCH₂CHa and SCH₂CHb), 1.20-1.43 (m, 6H, 3×CH₂), 0.87 (t, 3H, J 6.8 Hz, CH₃).

### EXAMPLE 8

Methods for the preparation of an alkyl thiol (HS—R₁) are well known to a person skilled in the art. In this case 11-heneicosanol was converted via a sulphonate ester (11-heneicosanyl p-toluenesulphonate) to the corresponding bromide (11-bromoheneicosane) before displacement by thioacetate anion to give 11-acetylthioheneicosane. The thioacetate was de-S-acetylated to give the desired thiol (11-heneicosanyl thiol). 11-Heneicosanol:

Magnesium turnings (705 mg, 28.9 mmol) and iodine (10 mg, catalytic) were combined and heated over a Bunsen flame under N₂ until I₂ gas evolved. The flask was allowed to cool, and then dry THF (100 mL) was added. Bromodecane (5.0 mL, 24.1 mmol) was added and the mixture was stirred for 2 h at 40° C. under N₂. After this time, undecylic aldehyde (5.0 mL, 24.1 mmol) was added and the reaction was stirred for a further 1 h, at 55° C., under N₂. The reaction was quenched with sat. aq. NH₄Cl, and the solvent was evaporated under reduced pressure. The residue was diluted with CH₂Cl₂ (300 mL) and extracted with aq. NaCl (200 mL) followed by water (200 mL). The organic layer was dried over Na₂SO₄, filtered, and solvent removed under reduced pressure. The residue was bonded to silica (dissolved in EtOAc and evaporated in the presence of silica) and chromatographed (hexane-DCM 2:1. TLC; R_{f} 0.57, Hex-EtOAc 6:1) to furnish 11-heneicosanol as a white powder (3.44 g, 46%). ¹H NMR (300 MHz, CDCl₃): δ 3.58 (m, 1H, OCHR₂), 1.26-1.48 (m, 36H, 18×CH₂), 0.88 (app t, 6H, J 6.9 Hz, 2×CH₃); ¹³C NMR (75.5 MHz, CDCl₃): δ 72.0 (CH), 37.5, 31.9, 29.7, 29.6, 29.3, 25.7, 22.7 (18×CH₂), 14.1 (2×CH₃); LRMS (ESI) m/z 335 [(M+Na)⁺ 8%] 413 (100) 489 (11).

### 11-Heneicosanyl p-toluenesulphonate

11-Heneicosanol (2.54 g, 8.14 mmol) and tosyl chloride (4.65 g, 24.4 mmol, 3 equiv.) were dissolved in dry pyridine (40 mL) at 0° C. under N₂. 4-Dimethylaminopyridine (10 mg, catalytic) was added and the mixture was stirred for 10 minutes at 0° C. under N₂. After this time the ice bath was removed and the mixture was stirred for a further 7 h at rt under N₂. The solvent was then removed under reduced pressure, and the residue dissolved in CH₂Cl₂. The solution was washed with 1 M HCl (100 mL), and then with sat. aq. NaHCO₃ (100 mL) to neutrality. The organic layer was dried over Na₂SO₄, filtered, and solvent removed under reduced pressure. The residue was chromatographed (hexane-EtOAc 20:1. TLC; R_{f} 0.63, hexane-EtOAc 12:1) to furnish 11-heneicosanyl p-toluenesulphonate as a clear oil (3.2 g, 88%). ¹H NMR (300 MHz, CDCl₃): δ 7.79 (d, 2H, J 8.3 Hz, SPhCH₃), 7.32 (d, 2H, J 8.0 Hz, SPhCH₃), 4.54 (quintet, 1H, J 6.0 Hz, CH), 2.44 (s, 3H, SPhCH₃), 1.49-1.62 (m, 4H, 2×CH₂), 1.09-1.37 (m, 32H, 16×CH₂), 0.88 (app t, 6H, J 6.8 Hz, 2×CH₃); ¹³C NMR (75.5 MHz, CDCl₃): δ 144.2, 134.8, 129.6, 127.7 (SPhCH₃), 84.6 (CH), 34.1, 31.9, 29.6, 29.5, 29.4, 29.3, 29.3, 24.7, 22.7 (18×CH₂), 21.6 (SPhCH₃), 14.1 (2×CH₃); LRMS (ESI) m/z 489 [(M+Na)⁺ 100%].

### 11-Bromoheneicosane

Lithium bromide (2.91 g, 33.5 mmol) and 11-heneicosanyl p-toluenesulphonate (2.20 g, 4.7 mmol) were dissolved in dry acetone (75 mL) and stirred at reflux for 3 h under N₂. After this time the solvent was removed under reduced pressure and the residue was dissolved in EtOAc (100 mL). The solution was washed once with aq. NaCl (100 mL) and once with water (100 mL), dried over Na₂SO₄, and the solvent removed under reduced pressure to furnish 11-bromoheneicosane as a clear oil (1.27 g, 72%). The brominated product was used without further purification. (TLC; R_{f} 0.87, hexane) ¹H NER (300 MHz, CDCl₃): δ 4.03 (tt, 1H, J_{A,B1=A,B′1} 5.7 Hz, J_{A,B2=A,B′2} 7.3 Hz, CH), 1.19-1.89 (m, 36H, 18×CH₂), 0.88 (app t, 6H, J 6.9 Hz, 2×CH₃); ¹³C NMR (75.5 MHz, CDCl₃): δ 59.1 (CH), 39.2, 31.9, 29.6, 29.5, 29.3, 29.1, 27.6, 26.5, 22.7 (18×CH₂), 14.1 (2×CH₃); LRMS (EI) 294 [(M−HBr) 100%] (HBr elimination).

### 11-Acetylthioheneicosane

11-Bromoheneicosane (1.00 g, 2.67 mmol) and potassium thioacetate (0.64 g, 5.80 mmol) were dissolved in dry acetone (50 mL) and refluxed for 20 h under N₂. After this time the solvent was removed under reduced pressure and the residue was dissolved in EtOAc (100 mL). The solution was washed once with aq. NaCl (100 mL) and once with water (100 mL), dried over Na₂SO₄, and the solvent removed under reduced pressure. The residue was chromatographed (hexane. TLC; R_{f} 0.33, hexane) to furnish 11-heneicosanyl thioacetate as a peach coloured oil (880 mg, 89%). ¹H NMR (300 MHz, CDCl₃): δ 3.50 (tt, 1H, J_{A,B1=A,B′1} 5.7 Hz, J_{A,B2=A,B′2} 7.6 Hz, CH), 2.31 (s, 3H, SAc), 1.15-1.65 (m, 36H, 18×CH₂), 0.87 (app t, 6H, J 6.7 Hz, 2×CH₃); ¹³C NMR (75.5 MHz, CDCl₃): δ 196.1 (COCH₃), 44.7 (CH), 34.8, 31.9 (2×CH₂), 30.8 (COCH₃), 29.6, 29.5, 29.5, 29.3, 26.8, 22.7 (16×CH₂), 14.1 (2×CH₃); LRMS (ESI) m/z 393 [(M+Na)⁺ 42%] 413 (100) 357 (42).

### 11-Heneicosanyl thiol

To a solution of 11-heneicosanyl thioacetate (500 mg, 1.35 mmol) in dry MeOH (30 ml) was added one ecuivalent of NaOMe (1.35 mL, 1M solution in dry MeOH). The reaction was stirred at rt for 70 minutes under N₂. After this time the solution was neutralised with Amberlite IR 120 (H⁺) resin, filtered and solvent removed under reduced pressure. The residue was dissolved in EtOAc (100 mL), washed once with ag. NaCl (100 mL), dried over Na₂SO₄, filtered and the solvent was removed under reduced pressure to furnish 11-heneicosanyl thiol as a pale yellow oil (330 mg, 75%). The de-S-acetylated product was used without further purification. (TLC; R_{f} 0.70, hexane). ¹H NMR (300 MHz, CDCl₃): δ 2.78 (m, 1H, CH), 1.19-1.70 (m, 36H, 18×CH₂), 0.88 (app t, 6H, J 6.9 Hz, 2×CH₃); ¹³C NMR (75.5 MHz, CDCl₃): δ 41.2 (CH), 39.0, 31.9, 29.6, 29.6, 29.4, 29.3, 27.1, 22.7 (18×CH₂), 14.1 (2×CH₃).

### 11-Heneicosanyl 2,3,5,6-tetra-O-benzoyl-1-thio-β-D-galactofuranoside (5, n=9)

To a solution of 1,2,3,5,6-penta-O-benzoyl-α/β-D-galactofuranose 1 (178 mg, 0.25 mmol) in dry CH₂Cl₂ (4 mL) was added tin (IV) chloride (32 μL, 0.28 mmol, 1.1 molar equiv.) and the solution was stirred for 10 minutes at 0° C. under N₂. 11-Heneicosanyl thiol (100 mg, 0.305 mmol, 1.2 molar equiv.) was then added and the reaction stirred for 2 h at 0° C. under N₂. After this time the solvent was removed under reduced pressure. The residue was then diluted in CH₂Cl₂ (50 mL) and extracted with sat. ag. NaHCO₃ (100 mL), dried over MgSO₄, filtered, and the solvent removed under reduced pressure. The residue was bonded to silica (dissolved in EtOAc and evaporated in the presence of silica) and chromatographed to give 5 (n=9) as a clear syrup (183 mg, 80%). R_{f} 0.60 (hexane-EtOAc 9:1); ¹H NMR (300 MHz, CDCl₃): δ 7.26-8.12 (m, 20H, 4×CO₂Ph), 6.10 (m, 1H, H-5), 5.65 (s, 1H, H-1) 5.64 (broad d, 1H, H-3 overlapped by H-1), 5.52 (app t, 1H, J_{2,3=2,1} 1.5 Hz, H-2), 4.83 (app t, 1H, J 4.6, J 4.1 Hz, H-4), 4.72 (m, 2H, H-6 and H-6′), 2.88 (m, 1H, SCHR₂), 1.62 (m, 4H, SCH(CH₂)₂), 1.15-1.51 (m, 32H, 8×CH₂, didecyl chain), 0.87 (m, 6H, 2×CH₃); ¹³C NMR (75.5 MHz, CDCl₃): δ 166.0, 165.7, 165.6, 165.4 (4×CO₂Ph), 133.5, 133.3, 133.2, 133.1, 130.1, 130.0, 130.0, 129.9, 129.7, 129.6, 129.5, 129.0, 129.0, 128.5, 128.4, 128.3, 128.3 (CO₂Ph), 87.8 (C-1), 83.1(C-2), 81.1 (C-4), 78.0 (C-3), 70.4 (C-5), 63.5 (C-6), 46.7 (SCHR₂), 35.4, 34.8, 31.9, 29.6, 29.6, 29.3, 26.9, 26.7, 22.7 (18×CH₂, didecyl chain), 14.1 (2×CH₃); LRMS (ESI) m/z 930 [(M+Na)⁺ 100%].

### EXAMPLE 9

### 11-heneicosanyl 1-thio-β-D-galactofuranoside (6, n=9)

11-Heneicosanyl 2,3,5,6-tetra-O-benzoyl-1-thio-β-D-galactofuranoside (5, n=9) was deprotected according to the general procedure. Yield: 74%. R_{f} 0.62 (EtOAc); ¹H NMR (300 MHz, CD₃OD): δ 5.02 (d, 1H, J 4.4 Hz, H-1), 3.99 (dd, 1H, J_{3,2} 4.8, J_{3,4} 7.5 Hz, H-3), 3.84 (dd, 1H, J_{4,3} 7.5, J_{4,5} 2.7 Hz, H-4), 3.82 (app t, 1H, J 4.4, J 4.7 Hz, H-2), 3.68 (dt, 1H, J_{5,4} 2.7, J_{5,6} 6.2, J_{5,6} 6.2 Hz, H-5), 3.55 (m, 2H, H-6 and H-6′), 2.72 (m, 1H, 2×SCHR₂), 1.53 (m, 4H, SCH(CH₂)₂), 1.17-1.47 (m, 32H, 8×CH₂, didecyl chain), 0.84 (t, 6H, J 6.6 Hz, 2×CH₃); ¹³C NMR (75.5 MHz, CD₃OD): δ 90.3 (C-1), 84.4 (C-2), 82.8 (C-4), 77.8 (C-3), 72.0 (C-5), 65.1 (C-6), 47.3 (SCHR₂), 36.6, 36.2, 33.1, 30.8, 30.7, 30.7, 30.5, 27.8, 23.8 (18×CH₂, didecyl chain), 14.5 (2×CH₃); LRMS (ESI) m/z 513 [(M+Na)⁺ 100%].

Biological Data

### EXAMPLE 10

Inhibition of various bacteria by compounds 4 (n=9) and 6 (n=9) is described in Table 1. The biological data were determined by minimum Inhibitory Concentration (MIC) Assays. Each compound was added to 4 ml LB broth at a starting concentration of 200 μg/ml. Serial dilutions were then made, 1 in 2 at each step, ending with 0.2 μg/ml. 5 μL of a saturated culture was added to each serial dilution, and incubated at 37° C. with shaking for 6 hours. The MIC was then determined as the lowest concentration showing inhibited bacterial growth.

TABLE 1

| Organism tested | Compound | MIC (μg/ml) |
|---|---|---|
| Mycobacterium smegmatis | 4 (n = 9) | <0.2 |
| | 6 (n = 9) | <0.2 |
| Staphylococcus aureus | 4 (n = 9) | <0.2 |
| | 6 (n = 9) | <0.2 |
| Bacillus subtilis | 4 (n = 9) | 25 |
| | 6 (n = 9) | >200 |
| Enterococcus faecalis | 4 (n = 9) | >200 |
| | 6 (n = 9) | 200 |

### EXAMPLE 11

Inhibition of various bacteria by compound 4 (n=9) is described in Table 2. The biological data were determined by a Zone Inhibition Assay method. Compound 4 (n=9) was tested by spotting 3 μL of a 10 μg/mL solution onto a filter disc which was placed on a lawn of bacteria on the surface of an LB agar plate. After incubation at 37° C. for three days (M. smegmatis) or overnight (other species), the zone of inhibition was measured using an arbitrary scale: +++=large zone of inhibition, −=no zone of inhibition.

TABLE 2

| Organism tested | Zone of Inhibition |
|---|---|
| Mycobacterium smegmatis | + |
| Staphylococcus aureus | + |
| Bacillus subtilis | + |
| Enterococcus faecalis | ++ |

### INDUSTRIAL APPLICABILITY

The compounds of general formula (I) are anti-microbial agents.

1. An anti-microbial compound of general formula (I): 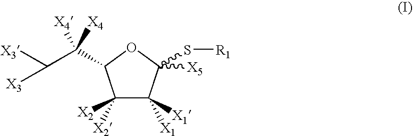
wherein R₁ is selected from the group consisting of alkyl, substituted alkyl, alkenyl, and substituted alkenyl, each of which is or is not interrupted by one or more heteroatoms or functional groups selected from the group consisting of O, S, —N═, NR₆ and —(Y)ₘC=(Z)(T)ₙ- and contains at least four carbon atoms, and aralkyl, substituted aralkyl which is or is not interrupted within the alkyl moiety by one or more heteroatoms or functional groups selected from the group consisting of O, S, —N═, NR₆ and —(Y)ₘC=(Z)(T)ₙ-;
X₁ is selected from the group consisting of OR₂, SR₂, NR₂R′₂, halogen, —(Y)ₘC=(Z)(T)ₙR₂, —N(C=(Z)(T)ₙR₂)₂, N₃, CN, OCN, SCN, OSO₃R₂, OSO₂R₂, OPO₃R₂R′₂, OPO₂R₂R′₂, S(O)R₂, S(O)₂R₂, S(O)₂OR₂, PO₃R₂R′₂, NNR₂R′₂, SNR₂R′₂, NHSR₂, SSR₂ and R₂, or is an oxo group, ═S, ═NOR₂ or ═CHR₂ and X₁′ is absent;
X₂ is selected from the group consisting of OR₃, SR₃, NR₃R′₃, halogen, —(Y)ₘC=(Z)(T)ₙR₃, —N(C=(Z)(T)ₙR₃)₂, N₃, CN, OCN, SCN, OSO₃R₃, OSO₂R₃, OPO₃R₃R′₃, OPO₂R₃R′₃, S(O)R₃, S(O)₂R₃, S(O)₂OR₃, PO₃R₃R′₃, NNR₃R′₃, SNR₃R′₃, NHSR₃, SSR₃ and R₃, or is an oxo group, ═S, ═NOR₃ or ═CHR₃ and X₂′ is absent;
X₃ is selected from the group consisting of OR₄, SR₄, NR₄R′₄, halogen, —(Y)ₘC=(Z)(T)ₙR₄, —N(C=(Z)(T)ₙR₄)₂, N₃, CN, OCN, SCN, OSO₃R₄, OSO₂R₄, OPO₃R₄R′₄, OPO₂R₄R₁₄, S(O)R₄, S(O)₂R₄, S(O)₂OR₄, PO₃R₄R′₄, NNR₄R′₄, SNR₄R′₄, NHSR₄, SSR₄ and R₄, or is an oxo group, ═S, ═NOR₄ or ═CHR₄ and X₃′ is absent;
X₄ is selected from the group consisting of OR₅, SR₅, NR₅R′₅, halogen, —(Y)ₘC=(Z)(T)ₙR₅, —N(C=(Z)(T)ₙR₅)₂, N₃, CN, OCN, SCN, OSO₃R₅, OSO₂R₅, OPO₃R₅R′₅, OPO₂R₅R′₅, S(O)R₅, S(O)₂R₅, S(O)₂OR₅, PO₃R₅R′₅, NNR₅R′₅, SNR₅R′₅, NHSR₅, SSR₅ and R₅, or is an oxo group, ═S, ═NOR₅ or ═CHR₅ and X₄′ is absent;
or X₁ and X₂ together constitute a double bond, or X₁ and X₂, X₂ and X₃, X₂ and X₄, X₃ and X₄, X₁ and X₁′, X₂ and X₂′, X₃ and X₃′ or X₄ and X₄′ together form a ring;
m and n are independently zero or one and Y, Z and T are independently selected from the group consisting of O, S, and NR₆ where R₆ is hydrogen or alkyl;
X₅, X₁′, X₂′, X₃′ and X₄′ are the same or different and are selected from the group consisting of hydrogen, CN, alkyl substituted alkyl, alkaryl substituted alkaryl, aryl, substituted aryl, aralkyl, and substituted aralkyl;
R₂, R′₂, R₃, R′₃, R₄, R′₄, R₅ and R′₅ are the same or different and are selected from the group consisting of hydrogen, alkyl substituted alkyl, alkaryl, substituted alkaryl, aryl substituted aryl, aralkyl substituted aralkyl, acyl substituted acyl and a carbohydrate moiety;
with the proviso that R₁ is other than benzyl and at least two of X₁, X₂, X₃ and X₄ are other than hydrogen or a group linked to the ring through a carbon-carbon bond;
or a pharmaceutically acceptable salt thereof.

2. A compound as claimed in claim 1 wherein R₁ is straight chain alkyl wherein a heteroatom or acyl linkage does or does not interrupt the chain.

3. A compound as claimed in claim 2 wherein R₁ is C₄₋₃₀ alkyl.

4. A compound as claimed in claim 1 wherein R₁ is a branched chain alkyl wherein a heteroatom or acyl linkage does or does not interrupt the chain.

5. A compound as claimed in claim 4 of general formula (II): 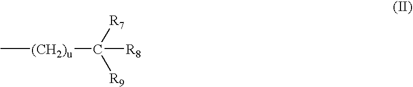
wherein u is 0 to 22 and wherein a heteroatom or acyl linkage may separate adjacent CH₂ groups within the moiety; and at least two of R₇, R₈ and R₉ are alkyl and the other is hydrogen or alkyl, and are the same or are different.

6. A compound as claimed in claim 5 wherein R₁ is a branched alkyl moiety of general formula (III). 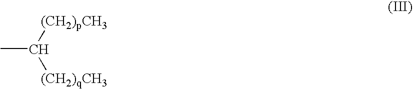
wherein p and q are the same or different and each is ≧1.

7. A compound as claimed in claim 1 wherein R₁ takes the general formula (IV): 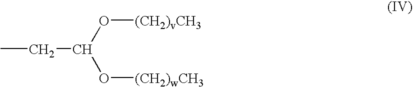
wherein v and w are the same or different and each is ≧1.

8. A compound as claimed in claim 1 wherein at least two of X₁, X₂, X₃ and X₄ are moieties linked to the ring through a carbon-oxygen bond.

9. A compound as claimed in claim 8 wherein X₁ is selected from the group consisting of OR₂, —(Y)ₘC=(Z)(T)ₙR₂ when Y is O, OSO₃R₂ and OPO₃R₂R₂′.

10. A compound as claimed in claim 9 wherein X₁ is hydroxyl or acyloxy.

11. A compound as claimed in claim 8 wherein X₂ is selected from the group consisting of OR₃, —(Y)ₘC=(Z)(T)ₙR₃ when Y is O, OSO₃R₃ and OPO₃R₃R₃′.

12. A compound as claimed in claim 11 wherein R₃ is hydroxyl or acyloxy.

13. A compound as claimed in claim 8 wherein X₃ is selected from the group consisting of OR₄, —(Y)ₘC=(Z)(T)ₙR₄ when Y is O, OSO₃R₄ and OPO₃R₄R₄′.

14. A compound as claimed in claim 13 wherein R₄ is hydroxyl or acyloxy.

15. A compound as claimed in claim 8 wherein X₄ is selected from the group consisting of OR₅, —(Y)ₘC=(Z)(T)ₙR₅ when Y is O, OSO₃R₅ and OPO₃R₅R₅′.

16. A compound as claimed in claim 15 wherein R₅ is hydroxyl or acyloxy.

17. A compound selected from the group consisting of:
hexadecyl 2,3,5,6-tetra-O-benzoyl-1-thio-β-D-galactofuranoside
decyl 2,3,5,6-tetra-O-benzoyl-1-thio-β-D-galactofuranoside
octyl 2,3,5,6-tetra-O-benzoyl-1-thio-β-D-galactofuranoside
hexyl 2,3,5,6-tetra-O-benzoyl-1-thio-β-D-galactofuranoside
11-heneicosanyl 2,3,5,6-tetra-O-benzoyl-1-thio-β-D-galactofuranoside
hexadecyl 1-thio-O-D-galactofuranoside
decyl 1-thio-β-D-galactofuranoside
octyl 1-thio-β-D-galactofuranoside
hexyl 1-thio-β-D-galactofuranoside and
11-heneicosanyl 1-thio-β-D-galactofuranoside.

18. (canceled)

19. (canceled)

20. A method for the treatment of a patient with a microbial infection, comprising administering to said patient a therapeutically effective amount of an anti-microbial compound of general formula (I) as claimed in claim 1.

21. A method of making a medicament for use in the treatment of a microbial infection, said method comprising providing an anti-microbial compound of general formula (I) as claimed in claim 1 and including a carrier, excipient, additive, or auxiliary.

22. A pharmaceutical composition comprising an anti-microbial compound of general formula (I) as claimed in claim 1 and a pharmaceutically acceptable carrier.

23. A method of killing a microorganism, comprising exposing said microorganism to an anti-microbial compound of general formula (1) as claimed in claim 1.

24. A compound as claimed in claim 3 wherein R₁ is C₆₋₁₆ alkyl.

25. A compound as claimed in claim 5, wherein u is 0 to 10.

26. A compound as claimed in claim 5, wherein u is 0 to 4.

27. A compound as claimed in claim 5, wherein u is 0 to 2.

28. A compound as claimed in claim 5, wherein u is 0.

29. A compound as claimed in claim 5, wherein alkyl is C₂₋₃₀ alkyl.

30. A compound as claimed in claim 5 wherein alkyl is C₆₋₁₆ alkyl, most preferably C₁₀ alkyl, and may be the same or different.

31. A compound as claimed in claim 5 wherein alkyl is C₁₀ alkyl.

32. A compound as claimed in claim 6 wherein each of p and q is 1-29.

33. A compound as claimed in claim 6 wherein each of p and q is 5-15.

34. A compound as claimed in claim 6 wherein each of p and q is 9.

35. A compound as claimed in claim 7 wherein each of v and w is 1-29.

36. A compound as claimed in claim 7 wherein each of v and w is 5-15.

37. A compound as claimed in claim 7 wherein each of v and w is 9.

38. A compound as claimed in claim 9 wherein —(Y)ₘC=(Z)(T)ₙR₂ is —OC(O)R₂.

39. A compound as claimed in claim 11 wherein —(Y)ₘC=(Z)(T)ₙR₃ is —OC(O)R₃.

40. A compound as claimed in claim 13 wherein —(Y)ₘC=(Z)(T)ₙR₄ is —OC(O)R₄.

41. A compound as claimed in claim 15 wherein —(Y)ₘC=(Z)(T)ₙR₅ is —OC(O)R₅.

42. A method of preparation of a compound of general formula (I): 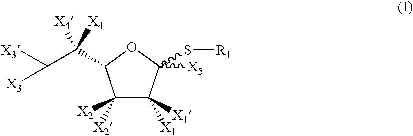
comprising reacting, in the presence of a base:
(a) a compound of general formula (V): 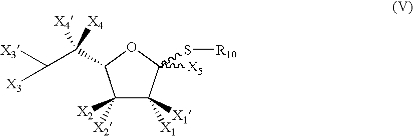
wherein R₁₀ is an acyl group, and
X₁ is selected from the group consisting of OR₂, SR₂, NR₂R₁₂, halogen, —(Y)ₘC=(Z)(T)ₙR₂, —N(C=(Z)(T)ₙR₂)₂, N₃, CN, OCN, SCN, OSO₃R₂, OSO₂R₂, OPO₃R₂R′₂, OPO₂R₂R′₂, S(O)R₂, S(O)₂R₂, S(O)₂OR₂, PO₃R₂R′₂, NNR₂R′₂, SNR₂R′₂, NHSR₂, SSR₂ and R₂, or is an oxo group, ═S, ═NOR₂ or ═CHR₂ and X₁′ is absent;
X₂ is selected from the group consisting of OR₃, SR₃, NR₃R₁₃, halogen, —(Y)ₘC=(Z)(T)ₙR₃, —N(C=(z)(T)ₙR₃)₂, N₃, CN, OCN, SCN, OSO₃R₃, OSO₂R₃, OPO₃R₃R′₃, OPO₂R₃R′₃, S(O)R₃, S(O)₂R₃, S(O)₂OR₃, PO₃R₃R′₃, NNR₃R′₃, SNR₃R′₃, NHSR₃, SSR₃ and R₃, or is an oxo group, ═S, ═NOR₃ or ═CHR₃ and X₂′ is absent;
X₃ is selected from the group consisting of OR₄, SR₄, NR₄R′₄, halogen, —(Y)ₘC=(Z)(T)ₙR₄, —N(C=(Z)(T)ₙR₄)₂, N₃, CN, OCN, SCN, OSO₃R₄, OSO₂R₄, OPO₃R₄R₁₄, OPO₂R₄R₁₄, S(O)R₄, S(O)₂R₄, S(O)₂OR₄, PO₃R₄R′₄, NNR₄R′₄, SNR₄R′₄, NHS₄, SSR₄ and R₄, or is an oxo group, ═S, ═NOR₄ or ═CHR₄ and X₃′ is absent;
X₄ is selected from the group consisting of OR₅, SR₅, NR₅R′₅, halogen, —(Y)ₘC=(Z)(T)ₙR₅, —N(c=(z)(T)ₙR₅)₂, N₃, CN, OCN, SCN, OSO₃R₅, OSO₂R₅, OPO₃R₅R′₅, OPO₂R₅R′₅, S(O)R₅, S(O)₂R₅, S(O)₂OR₅, PO₃R₅R′₅, NNR₅R′₅, SNR₅R′₅, NHSR₅, SSR₅ and R₅, or is an oxo group, ═S, ═NOR₅ or ═CHR₅ and X₄′ is absent;
or X₁ and X₂ together constitute a double bond, or X₁ and X₂, X₂ and X₃, X₂ and X₄, X₃ and X₄, X₁ and X₁′, X₂ and X₂′, X₃ and X₃′ or X₄ and X₄′ together form a ring;
X₅, X₁′, X₂′, X₃′ and X₄′ are the same or different and are selected from the group consisting of hydrogen, CN, alkyl substituted alkyl, alkaryl, substituted alkaryl, aryl, substituted aryl, aralkyl, and substituted aralkyl;
and wherein X₁, X₂, X₃ and X₄ have any free hydroxyl, thiol, or amine groups protected by a protecting group;
with (b) a compound of general formula (VI):
Br—R₁ (VI)
wherein R₁ is selected from the group consisting of alkyl, substituted alkyl, alkenyl, and substituted alkenyl, each of which is or is not interrupted by one or more heteroatoms or functional groups selected from the group consisting of O, S, —N═, NR₆ and —(Y)ₘC=(Z)(T)ₙ- and contains at least four carbon atoms, and aralkyl, substituted aralkyl which is or is not interrupted within the alkyl moiety by one or more heteroatoms or functional groups selected from the group consisting of O, S, —N═, NR₆ and —(Y)ₘC=(Z)(T)ₙ-; and
(c) wherein m and n are independently zero or one and Y, Z and T are independently selected from the group consisting of O, S, and NR₆ where R₆ is hydrogen or alkyl.

43. A method as claimed in claim 42 wherein R₁₀ is acetyl.

44. A method as claimed in claim 42, further including (d) removing the protecting groups.

45. A method of preparation of a compound of general formula (I): 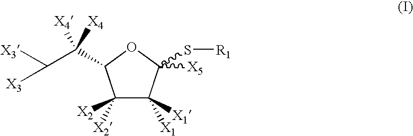
comprising reacting, in the presence of a catalyst:
(a) a compound of general formula (VII) 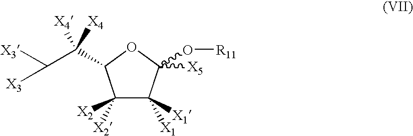
wherein R₁₁ is an acyl group, and
X₁ is selected from the group consisting of OR₂, SR₂, NR₂R′₂, halogen, —(Y)ₘC=(Z)(T)ₙR₂, —N(C=(Z)(T)ₙR₂)₂, N₃, CN, OCN, SCN, OSO₃R₂, OSO₂R₂, OPO₃R₂R′₂, OPO₂R₂R′₂, S(O)R₂, S(O)₂R₂, S(O)₂OR₂, PO₃R₂R′₂, NNR₂R′₂, SNR₂R′₂, NHSR₂, SSR₂ and R₂, or is an oxo group, ═S, ═NOR₂ or ═CHR₂ and X₁′ is absent;
X₂ is selected from the group consisting of OR₃, SR₃, NR₃R′₃, halogen, —(Y)ₘC=(Z)(T)ₙR₃, —N(C=(Z)(T)ₙR₃)₂, N₃, CN, OCN, SCN, OSO₃R₃, OSO₂R₃, OPO₃R₃R′₃, OPO₂R₃R′₃, S(O)R₃, S(O)₂R₃, S(O)₂OR₃, PO₃R₃R′₃, NNR₃R′₃, SNR₃R′₃, NHSR₃, SSR₃ and R₃, or is an oxo group, ═S, ═NOR₃ or ═CHR₃ and X₂′ is absent;
X₃ is selected from the group consisting of OR₄, SR₄, NR₄R′₄, halogen, —(Y)ₘC=(Z)(T)ₙR₄, —N(C=(Z)(T)ₙR₄)₂, N₃, CN, OCN, SCN, OSO₃R₄, OSO₂R₄, OPO₃R₄R′₄, OPO₂R₄R′₄, S(O)R₄, S(O)₂R₄, S(O)₂OR₄, PO₃R₄R′₄, NNR₄R′₄, SNR₄R′₄, NHSR₄, SSR₄ and R₄, or is an oxo group, ═S, ═NOR₄ or ═CHR₄ and X₃′ is absent;
X₄ is selected from the group consisting of OR₅, SR₅, NR₅R′₅, halogen, —(Y)ₘC=(Z)(T)ₙR₅, —N(C=(Z)(T)ₙR₅)₂, N₃, CN, OCN, SCN, OSO₃R₅, OSO₂R₅, OPO₃R₅R′₅, OPO₂R₅R′₅, S(O)R₅, S(O)₂R₅, S(O)₂OR₅, PO₃R₅R′₅, NNR₅R′₅, SNR₅R′₅, NHSR₅, SSR₅ and R₅, or is an oxo group, ═S, ═NOR₅ or ═CHR₅ and X₄′ is absent;
or X₁ and X₂ together constitute a double bond, or X₁ and X₂, X₂ and X₃, X₂ and X₄, X₃ and X₄, X₁ and X₁′, X₂ and X₂′, X₃ and X₃′ or X₄ and X₄′ together form a ring;
X₅, X₁′, X₂′, X₃′ and X₄′ are the same or different and are selected from the group consisting of hydrogen, CN, alkyl substituted alkyl, alkaryl, substituted alkaryl, aryl, substituted aryl, aralkyl, and substituted aralkyl;
and wherein X₁, X₂, X₃ and X₄ have any free hydroxyl, thiol, or amine groups protected by a protecting group;
with (b) a compound of general formula (VIII):
HS—R₁ (VIII)
wherein R₁ is selected from the group consisting of alkyl, substituted alkyl, alkenyl, and substituted alkenyl, each of which is or is not interrupted by one or more heteroatoms or functional groups selected from the group consisting of O, S, —N═, NR₆ and —(Y)ₘC=(Z)(T)ₙ— and contains at least four carbon atoms, and aralkyl, substituted aralkyl which is or is not interrupted within the alkyl moiety by one or more heteroatoms or functional groups selected from the group consisting of O, S, —N═, NR₆ and —(Y)ₙC=(Z)(T)ₙ-; and
(c) wherein m and n are independently zero or one and Y, Z and T are independently selected from the group consisting of O, S, and NR₆ where R₆ is hydrogen or alkyl.

46. A method as claimed in claim 45 wherein R₁₁ is acetyl or benzoyl.

47. A method as claimed in claim 45 wherein the catalyst is a Lewis acid.

48. A method as claimed in claim 45, further including (d) removing the protecting groups.
